(19) United States
(12) Patent Application Publication
(10) Pub. No.: US 2006/0293644 A1
(43) Pub. Date: Dec. 28, 2006

(54) System and methods for laser-generated ionizing radiation

(76) Inventor: Donald Umstadter, Lincoln, NE (US)

Correspondence Address: Charles C. Valauskas/Anuj K. Wadhwa;BANIAK PINE & GANNON, Suite 1200, 150 N. Wacker Drive, Chicago, IL, 60606, US

(21) Appl. No.: 11/471,158

(22) Filed: Jun. 20, 2006

Related U.S. Application Data
(60) Provisional application No. 60/692,472, filed on Jun. 21, 2005.
(60) Provisional application No. 60/702,347, filed on Jul. 25, 2005.

(51) Int. Cl. A61B 18/18

(52) U.S. Cl. 606/10; 606/15

(57) ABSTRACT

A system and methods for transporting light and then intensifying the light are provided. The system and methods can be utilized for a number of applications, including the detection and treatment of cancer and restenosis. Laser light is transported through a catheter (composed of capillaries) which can be fed into a patient allowing the end of the catheter to be placed in close proximity to a tumor. The laser light at the end of the catheter assembly can be reduced in pulse duration and focused to high intensity onto a target and thereby generate in vivo pulses of ionizing radiation.

This application claims the benefit of U.S. Provisional Application No. 60/692,472, filed Jun. 21, 2005, incorporated herein by reference in its entirety. This application also claims the benefit of U.S. Provisional Application No. 60/702,347 filed Jul. 25, 2005, incorporated herein by reference in its entirety.

### STATEMENT CONCERNING FEDERALLY SPONSERED RESEARCH

The development of this invention was in part supported by a government contract of the National Science Foundation grant number 0530778 and the Department of Energy grant number DEFG0205ER15663.

### FIELD OF THE INVENTION

The present invention relates to the field of catheter systems. More particularly, the present invention relates to the generation of ionizing radiation in order to image and/or treat patients. Even more particularly, the present invention relates to improving a catheter system to transport light in catheter systems and increase the intensity of a light pulse for the purpose of imaging and/or therapeutic applications. However, it is to be appreciated that the present invention is amenable to other like applications.

### BACKGROUND OF THE INVENTION

It is known in the art that catheter systems can be utilized in order to produce ionizing radiation. This can be accomplished via low-power, laser-triggered photo-cathodes that induce the breakdown of externally produced electric fields. The maximum energy of the radiation produced, however, is generally limited in this case, and only a limited number of types of radiation can be produced: electrons and x-rays. One known method to generate higher energy radiation of a greater number of types (electrons, x-rays, positrons, ions and neutrons) is through the use of much higher power lasers. See, e.g., U.S. Pat. No. 6,906,338, “Laser-driven ion accelerator,” and references therein, incorporated herein by reference. Indeed, it is also known to generate ionizing radiation with high energy using lasers based on chirped pulse amplification.

Capillaries can be utilized in order to transport light; however, high-power light is not transported as efficiently. See, e.g., U.S. Pat. No. 4,844,062, “Rotating fiberoptic laser catheter assembly with eccentric lumen,” and references therein, incorporated herein by reference. As such, it would be advantageous to transport laser light through a capillary and then intensify the light at the end of the catheter in order to create a high-power light that can used to generate ionizing radiation inside a patient's body.

Radiation can be used for various medical applications, for example, to treat cancer cells. The treatment of cancer cells represents one of the largest commercial markets, and it is expected to grow as the health-care markets in the developing world mature. As medical device technology moves towards greater reliance on minimally invasive imaging and therapeutic procedures, such uses of radiation are growing in importance. Moreover, radiation can be utilized in the treatment of restenosis. Restenosis is the renarrowing of a coronary artery after angioplasty. During a typical angioplasty, a stent is placed over the site of the artery blockage. Three to six months following the surgery, many patients suffer a buildup of scar tissue underneath the healthy tissue that has formed as a result of the stent. The growth of the scar tissue underneath the lining of the artery may grow to be so thick so as to cause blockage of the artery. Radiation treatment is a preventative application to the buildup of scar tissue and therefore is a preventative treatment of restenosis.

In vivo imaging directly with catheters using optical or infrared light optical coherence tomography has been proposed, U.S. Pat. No. 6,903,854, “Optical coherence tomography apparatus, optical fiber lateral scanner and a method for studying biological tissues in vivo,” but that is very different from using catheters to produce penetrating ionizing x-rays for imaging.

It would be advantageous to generate ionizing radiation with laser sources for use with internal medicine, or brachytherapy, applications. Many previous catheter systems that transported low-power light could not be used to produce ionizing radiation internal to the patient. Thus, previous methods utilized teletherapy, which suffers the disadvantage of damaging healthy tissue between the radiation source and the tumor.

It would also be advantageous to provide in vivo radiation generation as opposed to external radiation generation. Moreover, it would also be advantageous to provide significantly less collateral damage to healthy tissue compared to conventional therapies. It would also be advantageous to provide a therapy that does not have security issues with the storage of radio-isotopes. It would also be advantageous to provide detection with high resolution imaging. The present invention provides these advantages and solves the shortcomings of the prior art.

### SUMMARY OF THE INVENTION

In accordance with one aspect of the present invention, a catheter system for the generation of ionizing radiation is provided. The catheter system includes a laser source for generating a light pulse, a stretching element for receiving the light pulse to form a stretched light pulse and a catheter assembly. The catheter assembly includes a light input end including a light pulse spreading element, a catheter sleeve including a capillary bundle assembly and a light output end, wherein said light output end comprises a compression element to create a compressed light pulse; a multiple light pulse combining element; a focusing element; a renewable target; and an exit window.

In accordance with yet another embodiment of the present invention, said stretching element receives a light input from a flashlamp-pumped solid-state laser.

In accordance with still another embodiment of the present invention, said stretching element receives a light input from a diode-pumped solid-state laser.

In accordance with a further embodiment of the present invention, said capillary bundle assembly includes a plurality of capillaries.

In accordance with a further embodiment of the present invention, said light pulse spreading element receives the stretched light pulse and divides the stretched light pulse into multiple light pulses.

In accordance with a yet further embodiment of the present invention, said multiple light pulses are each distributed to a capillary within said capillary bundle assembly.

In accordance with another embodiment of the present invention, said capillary bundle assembly guides the light pulse from the light input end to the light output end.

In accordance with a still further embodiment of the present invention, each capillary within said capillary bundle assembly is coated with a high-damage-threshold material.

In accordance with a yet further embodiment of the present invention, said high-damage-threshold material is a dielectric.

In accordance with another embodiment of the present invention, said high-damage-threshold material is a metal.

In accordance with yet another embodiment of the present invention, said compression element includes at least one of a chirped window, a transmission grating, a Bragg grating, and a dispersive medium to compress the light pulse.

In accordance with another embodiment of the present invention, the multiple light pulse combining element includes a lens array to combine the compressed light pulse from each capillary within the capillary bundle assembly into a single light pulse having a diameter larger than any compressed light pulse within a single capillary.

In accordance with yet another embodiment of the present invention, said multiple light pulse combining element combines the multiple light pulses from each capillary within said capillary bundle assembly prior to the light pulses being compressed by the compression element.

In accordance with a further embodiment of the present invention, said focusing element increases intensity of said light pulse by focusing said light pulse.

In accordance with still another embodiment of the present invention, said renewable target is a capable of being ionized.

In accordance with a further embodiment of the present invention, said renewable target is a patient's tissue.

In accordance with yet another embodiment of the present invention, the stretching element stretches the light pulse in time, wherein said stretched light pulse has a linear chirp and the same frequency bandwidth as the light pulse from the light input.

In accordance with a still further embodiment of the present invention, a method for the treatment of non-healthy cells within a body having healthy and non-healthy cells is provided. The method comprises the steps of placing a catheter assembly within the body, said catheter assembly being part of a catheter system including a light source for generating a light pulse; a stretching element for receiving the light pulse to form a stretched light pulse; wherein said catheter assembly includes a light input end including a light pulse spreading element; a catheter sleeve including a capillary bundle assembly and a light output end, wherein said light output end comprises a compression element to create a compressed light pulse; a multiple light pulse combining element; a focusing element; a renewable target; and an exit window. The light source generates the light pulse to be received by the stretching element and stretching the light pulse and then spreads the stretched light pulse with the light pulse spreading element such that a portion of the stretched light pulse is dispersed to each capillary within the capillary bundle and is propagated through the capillary bundle assembly. Then, the light pulse from each capillary of the capillary bundle assembly is combined into a single light pulse with the multiple light pulse combining element to form a combined light pulse followed by compressing the combined light pulse within the catheter assembly with the compression element to create a compressed light pulse. The compressed light pulse is then focused on the renewable target, in order to create ionizing radiation. The ionizing radiation is then applied to the non-healthy cells.

In accordance with yet another embodiment of the present invention, a method for the detection of non-healthy cells within a body having healthy and non-healthy tissue is provided. The method comprises the steps of placing a catheter assembly within the body, said catheter assembly being part of a catheter system including a light source for generating a light pulse; a stretching element for receiving the light pulse to form a stretched light pulse; wherein said catheter assembly includes a light input end including a light pulse spreading element; a catheter sleeve including a capillary bundle assembly and a light output end, wherein said light output end comprises a compression element to create a compressed light pulse; a multiple light pulse combining element; a focusing element; a renewable target; and an exit window. The light source generates the light pulse to be received by the stretching element and stretching the light pulse and then spreads the stretched light pulse with the light pulse spreading element such that a portion of the stretched light pulse is dispersed to each capillary within the capillary bundle and is propagated through the capillary bundle assembly. Then, the light pulse from each capillary of the capillary bundle assembly is combined into a single light pulse with the multiple light pulse combining element to form a combined light pulse followed by compressing the combined light pulse within the catheter assembly with the compression element to create a compressed light pulse. The compressed light pulse is then focused on the renewable target, in order to create ionizing radiation. The ionizing radiation is then applied within the body in conjunction with an external detector in order to determine whether the tissue in said body is non-healthy or healthy.

In accordance with a further embodiment of the present invention, the non-healthy cells are cancerous cells.

In accordance with a still further embodiment of the present invention, the non-healthy cells are scar tissue formed by restenosis.

In accordance with another aspect of the present invention, a system for detecting structural damage in a structure is provided. The detection system includes a laser source for generating a light pulse, a stretching element for receiving the light pulse to form a stretched light pulse and a detection assembly. The detection assembly includes a light input end including a light pulse spreading element, a detector sleeve including a capillary bundle assembly and a light output end, wherein said light output end comprises a compression element to create a compressed light pulse; a multiple light pulse combining element; a focusing element; a renewable target; and an exit window.

In accordance with still another embodiment of the present invention, a method for the detection of structural damage within a structure is provided. The method comprises the steps of placing a detection assembly within the body, said detector assembly being part of a detection system including a light source for generating a light pulse; a stretching element for receiving the light pulse to form a stretched light pulse; wherein said detection assembly includes a light input end including a light pulse spreading element; a detector sleeve including a capillary bundle assembly and a light output end, wherein said light output end comprises a compression element to create a compressed light pulse; a multiple light pulse combining element; a focusing element; a renewable target; and an exit window. The light source generates the light pulse to be received by the stretching element and stretching the light pulse and then spreads the stretched light pulse with the light pulse spreading element such that a portion of the stretched light pulse is dispersed to each capillary within the capillary bundle and is propagated through the capillary bundle assembly. Then, the light pulse from each capillary of the capillary bundle assembly is combined into a single light pulse with the multiple light pulse combining element to form a combined light pulse followed by compressing the combined light pulse within the detector assembly with the compression element to create a compressed light pulse. The compressed light pulse is then focused on the renewable target, in order to create ionizing radiation. The ionizing radiation is then applied within the structure in conjunction with an external detector in order to detect whether there is structural damage in a structure.

### BRIEF DESCRIPTION OF THE DRAWINGS

FIG. 1 illustrates an exemplary embodiment of a catheter system in accordance with the teachings of the present invention.

FIG. 2 illustrates a graph showing the absorbed dose versus the depth in tissue of radiation on the way from outside the patient's body to the tumor, as would be the case with teletherapy.

### DETAILED DESCRIPTION OF AN EMBODIMENT OF THE INVENTION

In accordance with one embodiment of the present invention, a system and methods for laser-generated ionizing radiation are provided for, among other applications, imaging and/or therapeutic applications. Embodiments in accordance with the present invention allow for the local production of laser generated ionizing radiation, which can be advantageously used in medical applications, such as the detection and treatment of cancerous cells and also the treatment of restenosis. Although described in detail with respect to one embodiment of the present invention, it is to be appreciated that the teachings of the present invention are amenable to other applications, as described below.

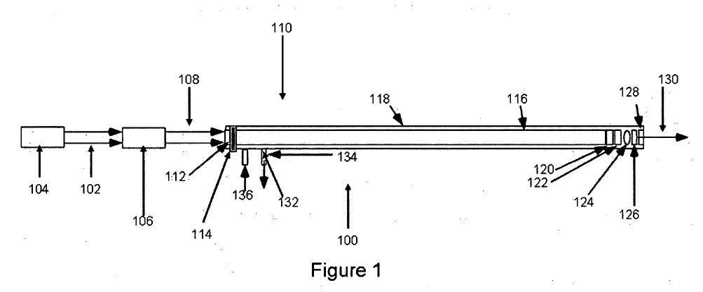

FIG. 1 shows one embodiment of a catheter system 100 in accordance with the present invention for producing laser-generated ionizing radiation. As can be seen, a light input (or light pulse) 102 from a light source 104 is stretched by a stretching element 106 to form a stretched light input 108. The stretched light input 108 is then sent to the catheter assembly 110, which receives the stretched light input 108 (typically a light pulse) from the laser source 104 through the catheter assembly entrance window 112. The stretching element 106 preferably includes dispersive (in time) features, such as gratings prisms, or optical fibers (See M. Pessot, P. Maine, and G. Mourou, “1000 Times Expansion/Compression of Optical Pulses for Chirped Pulse Amplification,” Opt. Comm. 62:419 (1987)). Also preferably included in catheter systems in accordance with the present invention is a light pulse spreading element 114, which in some embodiments, includes a lens array. After the light pulse has been spread, portions of the light input will disperse (in space) by the light pulse spreading element 114 so as to allow the light input to be distributed to each capillary within the capillary bundle assembly 116. The capillary bundle assembly 116 preferably includes a plurality of capillaries and is encased in a catheter sleeve 118. Thus, the light pulse spreading element 114 takes the single laser pulse (or beam) and breaks the single beam into many smaller beams (not shown), each of which propogates through a capillary (not shown) within the capillary bundle assembly 116. Upon traveling through the capillary bundle assembly 116, the light input from each capillary within the capillary bundle assembly 116 is preferably combined by the multiple light pulse combining element 120, to form a combined light pulse (not shown). After being combined, the combined light pulse is preferably compressed by the compression element 122. In some embodiments, the multiple light pulses are compressed individually prior to being combined by the multiple light pulse combining element 120. In some embodiments, the combined light input is “chirped” at a chirped window, which is one example of how a combined light input can be compressed. The combined light input preferably has a diameter that is greater than the diameter of any single light pulse from a single capillary within the capillary bundle assembly 116. The combined light input (not shown) then travels through a focusing lens 124 (which is one way in which the combined light input can be intensified by self-focusing) to the renewable target 126. In alternative embodiments, a lens array may be utilized to intensify the combined light input. When contacting the renewable target 126, the intensified light pulse generates ionizing radiation 130, which is released from the catheter assembly 110 through the exit window 128. In certain embodiments, as further explained below, the renewable target 126, may be located outside of the catheter assembly 110 and the ionizing radiation 130 may contact the renewable target following exiting the catheter assembly 110 through the exit window 130. The exit window 128 can also act to filter out certain unwanted frequencies of the ionizing radiation 130. Preferably, as explained below, certain embodiments in accordance with the present invention include a vacuum pump outlet 132 which can be attached to the catheter assembly 110 through vacuum tubing 134, and also include a gas inlet 136.

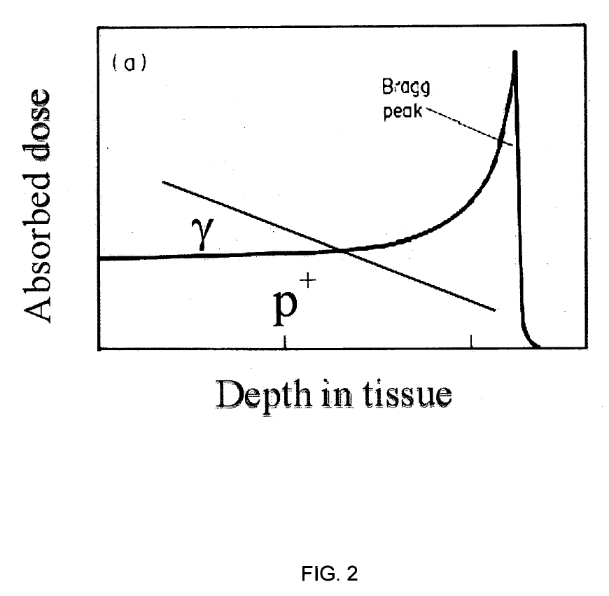

When a terawatt laser source is focused to high intensity, it has been shown to generate large fluxes of ionizing radiation (electrons, positrons, protons, ions, or x-rays). See, Umstadter, D., “Topical Review: Relativistic Laser Plasma Interactions,” J. Phys. D: Appl. Phys. 36, R151-R165 (2003), the teachings of which are incorporated herein by reference. These types of radiation have also been produced by more conventional means, such as with radio-frequency-based accelerators or radioactive isotopes. These types of radiation have also been shown to detect (through imaging), or destroy (through therapy) cancerous cells. One problem encountered when using these types of radiation when they are produced external to the patient (teletherapy) is that they kill healthy tissue as they are transported through the body to the location of the tumor. As shown in the FIG. 2 chart 200, the x-axis 202 represents the depth in tissue and the y-axis 204 represents the absorbed dose. As shown in the chart 200, most of the radiation that is absorbed by healthy tissue on the way from outside the patient's body 206 to the tumor 208.

Transportation of externally-produced radiation without damaging healthy tissue is also difficult once the radiation is generated as is the case for teletherapy. Thus, short-lived isotopes have been transported into the body, where they generate radiation internally, as in brachytherapy. It is advantageous to produce the radiation locally to the area that desired to be treated, either for detection and/or treatment of cancerous cells or for the treatment of restenosis. Diagnosis of cancerous tumors can also be accomplished in this way, when the detector is external, as in nuclear medicine procedures such as positron-emission tomography (brachytherapy). In accordance with embodiments of the present invention a system and methods for transporting laser light into a patient through a catheter assembly composed of a capillary bundle assembly, or by other optical means, is provided where the radiation can be focused to be of high intensity, thereby generating in vivo pulses of ionizing radiation. Radiation formulated in accordance with the present invention can be used for the purpose of detecting (or treating) cancer or restenosis by means of imaging (or brachytherapy).

Terawatt laser sources for use in embodiments of the present invention can be based on chirped pulse amplification, where the chirped window is preferably included in a compression element. Laser sources for use with embodiments of the present invention are preferably diode- or flashlamp-pumped solid-state laser sources with typical parameters of 20-100 fs pulse duration and 50-250 mJ energy per pulse, and operate at a 10-1,000 Hz repetition rate. A gas laser, such as one based on carbon dioxide, preferably with a shortened pulse duration (such as a fast electro-optic switch), is also contemplated for use with embodiments in accordance with the present invention. In other embodiments in accordance with the present invention, active phase control could be incorporated into the system by means of a “Dazzler,” or other spatially resolved phase modulator system as known by those skilled in the art. Thus, the output of the laser system is preferably the input of the capillary/catheter system.

Typical catheter systems employ fiber optics, which can only transport low power light. One disadvantage is that low power light is incapable of generating ionizing radiation when focused. High-power (terawatt) light sources, however, cannot be transported through the usual fiber optics, because nonlinear optical effects damage the optical elements. Capillary fibers, however, have been shown to be capable of transporting high-power light sources. Embodiments in accordance with the present invention preferably employ a catheter system including a flexible, evacuated capillary bundle assembly, with a lens or di-electric coated mirror at its end, which can be used to focus the laser light onto a target (see FIG. 1). It is contemplated that different capillary types could be used in accordance with the teachings of the present invention.

The term “capillary” in accordance with the teachings of the present invention refers to a class of low-transmission loss optical devices that includes hollow-core fibers, or other hollow fibers or waveguides. Capillaries for use in accordance with the present invention typically can be composed of flexible (bendable) dielectric or metallic material, e.g., either glass, sapphire, silver, gold, or plastic. Preferably, a plurality of capillaries is included in the capillary bundle assembly. The walls of such optical fibers or capillaries (waveguides) are preferably coated with a high-damage-threshold material, composed of dielectric (e.g., polymer) or metal (e.g., silver or gold). Although this is the preferred means, it should not be taken to exclude the use of other types of non-hollow fiber optics. It is contemplated that, if necessary, at high-average power, a cooling system may be utilized in order to remove the heat generated by the laser. The fiber(s) might themselves be, or be coupled to, a pulsed high-power fiber laser(s). In this case, the fiber would be filled with an active material and pumped by multi-mode pump light from semiconductor laser diodes.

The capillary bundle assembly includes a plurality of individual capillaries and is preferably evacuated by a vacuum pump. The target feed system could be a separate apparatus, but is preferably part of the catheter system. The pressure in the capillaries is preferably monitored with a vacuum gauge. The stretched light input, for example from a laser source, preferably enters the beginning of the capillaries through a thin window and exits the capillaries through the multiple light pulse combining elements, light pulse compression elements and lens. Some catheter assemblies preferably include positioning control systems, such as a lamp, CCD camera and monitor.

Targets utilized in catheter systems described in embodiments of the present invention are preferably composed of a renewable thin layer of solid, liquid, or gas, which become ionized by the light input and emit ionizing radiation. Preferably, the target's thickness, density, composition, surface roughness and other variables can all be adjusted to maximize the efficiency, i.e., the amount and properties of the radiation emitted with the minimum incident laser energy. Although an adjustable target is preferred, it is contemplated that the patient's own tissue could be used as the target material. Under this embodiment, the light input (or light source) could impinge directly on the patient's tissue, creating a plasma that radiates ionizing radiation. In yet other embodiments in accordance with the present invention, the ionizing radiation that is produced by the focused laser source preferably originates from radio-isotopes that are generated though nuclear activation. In such embodiments, a separate activation target is preferably utilized, or, alternatively, the tissue of the patient might again serve as the activation target.

In yet another embodiment in accordance with the present invention, the renewable target or medium is alternatively a replaceable section of the cathteter assembly. One advantage in this embodiment of the present invention is that the replaceable section of the catheter assembly assembly could be damaged during a given treatment session and the easily replaced between uses (such as prior to the follow-on treatments). The replaceable section could be any portion of the catheter assembly, including the target section, the focusing section, the compressing section, the multiple light pulse combining section or any combination of any sections of the capillary bundle assembly.

In yet another embodiment in accordance with the present invention, a lens array for increasing the intensity of the laser light input or source is provided. Alternatively, a single lens may be self-focusing in the fiber optical capillary in order to increasing the intensity of the laser light input. Preferably, the laser light input is intensified following re-combination of the stretched light input or compression of the combined light pulse. Self-focusing in accordance with this embodiment of the invention can be achieved either due to the atomic susceptibility of a neutral medium or from relativistic effects in an ionized media (such as, for example, plasma), which is created when the laser reaches an intensity that exceeds the ionization threshold. The ionization threshold is such that would be appreciated by those skilled in the art. The medium can be either within the capillary tube or be the target material at the end of the capillary bundle assembly. It is further contemplated that some self-focusing may occur naturally if the compression of the pulse duration is also accomplished by means of dispersion in a medium. It is also contemplated that self-focusing may be induced by tailoring the profile of the medium, such as, for example, is the case for a section of fiber optic with a radial index-of-refraction gradient. It is therefore contemplated that the value of the radial index-of-refraction gradient may require variations along the length of the fiber.

When a bundle of fibers, i.e., within the capillary bundle assembly, is used to transport a preferred amount of laser energy from the laser source, embodiments in accordance with the present invention optically demagnify and up collimate the stretched light input exiting the fibers (e.g., with a lens array), such that the aperture of the light is as large as possible before entering the final focusing optic, making the f/# of the final focusing optic as small as possible, and thus making it possible to generate the highest possible intensity on the target with the minimum energy from the light source. One way this can be achieved is through the utilization of an active deformable mirror (containing actuators that can locally deform the mirror's surface to adjust the light pulse's phase fronts) which may also be employed (before the stretched light input enters the capillary bundle assembly), and compensate (and pre-compensate) the transverse spatial phase distortions from the laser source itself (and from the optical elements in the catheter system).

Both the index-of-refraction (dispersion) and the phase profile of the light source are controllable in such a manner as to accomplish both compression and focusing in order to achieve the highest possible intensity at the end of the capillary bundle assembly, which may correspond with the location of the target. Control of the index-of-refraction and the phase profile of the light source can be achieved in many ways, including with a pre-compensating phase control system (such as a “Dazzler”), by use of an index-of-refraction gradient, a phase grating, dispersion in a medium (preferably within either the capillary or the target) or by a combination of any of the above methods or any other method as would be appreciated by those skilled in the art. Those skilled in the art would also recognize that the balance between compression and focusing may be adjusted in order to be effective for a particular application.

Preferably also included in the catheter system is a window at the end of the capillary bundle assembly that can preferably be “chirped” (or compressed), such that it would compress the light pulse after the stretched light input has propagated down the capillary bundle assembly and combined using the multiple light pulse combining element. This is similar to chirped mirror technology, but for transmission rather than reflection. It is contemplated, however, that a transmission grating, or a fiber Bragg grating, or a dispersive medium, may also serve the same function. The light pulse might also be pre-compensated for the extra phase accumulated after the compression stage with a phase modulator so that the pulse of light becomes short only when it penetrates (or reaches) the target material. The spatial-phase modulator and dispersion through the fiber and/or gas within the fiber, and/or target might be sufficient for active compression of the light pulse without the use of any compressor stage, or grating (N. Karasawa, L. Li, A. Suguro, H. Shigekawa, R. Morita, and M. Yamashita, “Optical pulse compression to 5.0 fs by use of only a spatial light modulator for phase compensation,” J. Opt. Soc. Am. B 18, 1742-1746 (2001)). This might also be done adaptively (D. Yelin, D. Meshulach, and Y. Silberberg, “Adaptive femtosecondpulse compression,” Opt. Lett. 22, 1793-1795 (1997)). Embodiments in accordance with the present invention featuring this characteristic may eliminate the need for vacuum conditions in the fiber or capillary (capillary bundle assembly). Thus, a light pulse with a longer pulse duration and lower peak power could be propagated down the capillary bundle assembly than would otherwise be possible, thereby reducing the damage to the walls of each capillary within the capillary bundle. It is contemplated that any number of means for combining and then compressing the light pulse could be utilized in embodiments of the present invention as would be appreciated by those of ordinary skill in the art.

The term “chirping” typically refers to a technique for the modulation of laser pulses emitted from a coherent optical light source emerges from similarly designated, partially analogous techniques originally developed in connection with radar scanning devices, wherein outgoing atmospheric signal ranging pulses are typically frequency modulated, increasing (or decreasing) the signal frequency within each emitted pulse, to produce a repetitive stream of variable frequency pulses, similar in that sense to the patterned type of tonal pitch variations that may be heard from birds or crickets (therefore the term “chirping”).

As applied within the context of optical laser technology, the term “chirping” most often defines a modulation/demodulation process for optical pulse transmission wherein the duration of each discretely emitted pulse is first expanded, usually by bracketing the coherent optical beam between a pair of diffraction grating filters, co-aligned in precise orientation, to obtain a linear devolution of spectral frequency components making up each discrete optical pulse, producing thereby a spatial/temporal stretching of the pulsed emissions, which also reduces the peak amplitude of each separate pulse. Chirped pulse amplification techniques for producing ultra-short, high-intensity optical laser pulse emissions were the subject of patents issued during the early 1990's by Mourou et al. (U.S. Pat. Nos. 4,918,751 and 5,235,606).

Instead of using ordinary diffraction gratings, it is also possible to use Bragg-type filter gratings, having additional bandpass filter capabilities (for example, as described in U.S. Pat. No. 5,499,134). Demodulation is then accomplished in a second step, conventionally again by bracketing the stream of optical laser pulses between a second pair of diffraction grating filters, to recompress the spectral components of the pulsed waveforms, thus to restore the coherent optical laser beam.

The technical basis for optical pulse dispersion/compression has been reviewed by Treacy (E. B. Treacy, “Optical Pulse Compression with Diffraction Gratings,” IEEE J. Quantum. Elect. 5:454-458 (1969)), and proposed for adaptation to optical communications technology by Martinez (O. E. Martinez, “Design of high-power ultrashort pulse amplifiers by expansion and recompression,” IEEE J. Quantum Electr. 23:1385-1387 (1987)). Similar techniques were shown to have useful practical application in the propagation of signal pulses transmitted through optical fibers (U.S. Pat. Nos. 4,655,547; 4,928,316; and 5,113,278), including for example optical communication systems using code division multiple access (CDMA) technologies (U.S. Pat. No. 4,866,699).

An amplification step may be optionally interposed between signal dispersion and recompression. As known by those skilled in the art, amplification of an optical laser input may be obtained from optical pumping of Ti:sapphire, Nd:YAG, Nd:glass, Yb:silica, Alexandrite, or other such high-energy-storing crystalline material. Photons from multiply impinging light sources arriving with precisely tuned phase conjugation may be transiently stored within the atomic matrix of the crystalline target structure, to a material-specific point of saturation, triggering then a process of stimulated emission, with resulting amplification of the emitted beam intensity. Pessot et al. (M. Pessot, P. Maine, and G. Mourou, “1000 Times Expansion/Compression of Optical Pulses for Chirped Pulse Amplification,” Opt. Comm. 62:419 (1987)) demonstrates that, by careful arrangement and exact calibration of the diffraction grating filter pairs, the tandem processes of dispersion/compression could be made fully complementary and precisely opposite in their effects, meaning that amplification might occur without causing any substantial amount of signal distortion within the subsequently recollimated stream of optical pulses. See also, U.S. Pat. No. 4,918,751 to Pessot et al., the teachings of which are incorporated herein by reference.

The aggregate intensity of a single discrete optical light pulse having sub-nano Joule energy may typically be increased by a factor of 10³ or more during the stretching devolution step utilizing the stretching element, and then enhanced by a factor of about 10¹¹ from stimulated emission using presently available amplification techniques. Reconstituting the spectral components of the optical pulse provides an additional intensification increment, by concentrating the pulse energy within a very narrowly spiked, ultra-short pulse period. As a result of this process, the energy intensity of an ultra-short pulse lasting only 10 or 20 femtoseconds may then be measured in the range of terawatts per square centimeter. With further technological improvements, it has been shown that generation of light pulses having petawatt per square centimeter and exawatt per square centimeter intensities have been obtained.

It is known by those of ordinary skill in the art that light pulses with energies of several milli-Joules can propagate down capillaries of lengths of tens of centimeters. A bundle of several such capillaries, such as in the capillary bundle assembly, enables terawatt power levels to be brought through the catheter system, which could be focused to intensities reaching at least 10ˆ18 W/cmˆ2, sufficient to generate copious amounts of ionizing radiation. The light pulse from the laser source typically can be coupled into the fiber by utilization of a focusing lens or mirror. To prevent air breakdown, the region between the focusing lens and the capillary waveguide would either be evacuated or contain an air pressure or high-breakdown-threshold dielectric (such as sulfur hexafluoride) that has been adjusted to prevent breakdown at the highest possible laser fluence. The same measures to prevent air breakdown could also be employed in other areas of the catheter system, for example, in the region of the waveguide, between the input and the target, instead of the evacuation of air, as discussed above.

In addition, it is contemplated that the laser source parameters, such as pulse duration, can be adjusted to minimize air breakdown. It is to be appreciated by those skilled in the art that other means to prevent air breakdown could also be implemented in accordance with the teachings of the present invention and that such means could be implemented in preferred areas throughout the assembly. For example, tapering the bore diameter of an individual capillary within the capillary bundle assembly, might increase its light coupling efficiency and also reduce the damage to each of the capillary entrance walls. Moreover, it is contemplated that the laser source parameters, such as wavelength, mode quality, pulse duration and other parameters, can be varied to increase coupling efficiencies, e.g., the coupling into the capillary waveguide and conversion to ionizing radiation at the target.

It is known by those of ordinary skill in the art that light can be compressed by means of spectral broadening in hollow fibers that are filled with a noble gas, such as krypton, followed by compression in a dispersive system (M. Nisoli, S. De Silvestri, O. Svelto, R. Szipcs, K. Ferencz, C. Spielmann, S. Sartania, and F. Krausz, “Compression of high-energy laser pulses below 5 fs,” Opt. Lett. 22, 522-524 (1997)). The spectral broadening arises from self-phase modulation (due to nonlinear interaction of the light with the gas). It is contemplated that this principle might be used to produce shorter pulses and thus higher intensities at the capillary output.

The catheter system can be fed into the patient so that its end is placed in close proximity to the tumor or artery in the case of restenosis. A detector external to the patient could be used to detect the radiation, and thus create an image of the tumor, an image with much higher resolution (tens of microns) than produced with conventional teletherapy or positron-emission tomography. It is contemplated that both the light source and the stretching element may be separate from the catheter assembly, so as to minimize the diameter of the catheter system and allow for more precise placement of the catheter system in the patient to be placed as close as possible to the tumor for cancerous tumor treatment or artery in the case of treatment for restenosis. As new light sources and stretching elements on a smaller scale are developed (such as those made of fibers), it is contemplated that catheter systems in accordance with the present invention may include light sources and stretching elements in the catheter assembly. In other embodiments in accordance with the present invention, a radiation detector (preferably spatially and/or spectrally resolving), such as an x-ray charge-coupled device, could be placed within the patient with the same or another catheter. Such embodiments would allow for lower doses and photon energies of radiation to be used than in the case of external detectors, and would also dramatically improve the image resolution. Such embodiments may also enable in situ detection of cancer tissue spectroscopically by means of absorption spectroscopy, since there would be very little healthy tissue between the detector and the radiation source as compared with conventional techniques. Alternatively, the radiation could be produced with sufficient flux to kill cancer cells, as in conventional brachytherapy, or sufficient for the treatment

Positioning of the catheter system might also be accomplished by operation in a low power mode, whereby low level radiation is generated at the end of the catheter system, the position of which can be spatially resolved by external detectors. The catheter assembly can be rotated, as is the case with conventional laser source catheters, so that an area that is larger than the catheter tip (the end of the catheter assembly where ionizing radiation is outputted) can be exposed.

Radiation with short range in tissue, such as ions, produces very little collateral damage to healthy tissue, such as in Boron-neutron capture therapy, as recognized by those skilled in the art. The range of a 70 MeV proton is approximately a centimeter, the size of the average treatable tumor. Radiation emitted such that it has an intensity that drops quadratically with radius (1/rˆ2) (such as gamma rays) would also deposit more of their energy in at the location of the tumor site than in the surrounding healthy tissue. Previously, since proton therapy employed protons that were produced external to the patient, the minimum proton energy was 280 MeV, which is preferred in order to penetrate through about 10 centimeters of the patient's body from outside the location of the tumor. The achievement of such a high level of energy has typically required the use of large, massive and expensive conventional radio-frequency accelerators. However, by lowering the required proton energy, embodiments in accordance with the present invention enable proton therapy with laser-driven protons (which have already produced energies up to 60 MeV), allowing for the reduction in size and cost of the accelerator, while also improving the efficacy of proton therapy by reducing the damage to healthy tissue. It is contemplated that the same principles would apply to the use of other ions in hadron therapy as would be appreciated by those skilled in the art. Thus, much less collateral damage to healthy tissue would be produced than with conventional teletherapy with gamma rays, where the radiation source is outside of the patient's body. The dose can also be significantly higher than from radio isotope decay, and none of the security issues associated with the storage of radio-isotopes exists. Thus, it is one advantage of the present invention to produce radiation locally to the area of treatment, so as to avoid collateral damage to healthy tissue.

In accordance with still other embodiments of the present invention, the application of brachytherapy with laser generated ionizing radiation can be used for the treatment of restenosis. Restenosis, which may follow a coronary angioplasty, is a problem facing percutaneous coronary interventions, despite advances in stent designs and new antiplatelet therapies. Recent studies have shown that endovascular radiation can limit the formation of neointimal tissue in the vascular wall and appears to be a promising method to control the retenotic process. See J. C. Blanco, et al., Rev. Esp. Cardiol. 1997 July; 50(7)-520:8, whose teachings are incorporated herein by reference. Catheter systems in accordance with the present invention can be inserted into the artery and used to apply radiation, either x-rays or charged particles). One advantage of using catheter systems in accordance with the present invention is that the laser source-driven radiation is preferably to alternative approaches because of the larger range of both the types and the energies of radiation that can be applied. Another advantage of catheter systems in accordance with the present invention is that not only is the radiation generated locally, to avoid collateral damage to healthy cells and vessels, but also that the user of the catheter may apply the tip of the catheter along the artery, so as to “trace” the artery and provide treatment as close to the affected area as possible. Furthermore, use of ionizing radiation in accordance with the present invention can be preventative to debris and other cell buildup (for example, scar tissue) after treatment for arterial sclerosis. It is to be appreciated to those skilled in the art that the disclosed system and methods are not to be restricted to the treatment of cancer and restenosis, but is contemplated to include any disease that is treatable with ionizing radiation, either in humans or small mammals.

It is contemplated that catheter systems in accordance with the present invention can also be utilized for various commercial applications, including, but not limited to, oncological, surgical, cardiovascular (such as stenosis and restenosis), urological procedures, gynecological procedures, veterinary, therapeutic, medical imaging and various potential non-medical applications, such as materials imaging. Other applications will be appreciated by those skilled in the art upon a reading of the present disclosure.

Various applications can be utilized with catheter systems in accordance with the present invention, including detecting solid-mass tumors with high-energy lasers, treating solid-mass tumors using high-energy lasers, detection of cracks and structural damage with high-intensity lasers, catheter systems for high-intensity light sources (preferably with a capillary waveguide), proton radiation therapy using a high-energy light sources and treatment of cardiac restenosis using a high-energy, ultra-short laser, to name a few applications.

The invention has been described with reference to preferred embodiments. Obviously, modifications and alterations will occur to others upon a reading and understanding of this specification. It is intended that the invention be construed as including all such modifications and alterations insofar as they come within the scope of the appended claims or the equivalents thereof.

What is claimed is:

1. A catheter system for generation of ionizing radiation, comprising
a laser source for generating a light pulse;
a stretching element for receiving the light pulse to form a stretched light pulse;
a catheter assembly including
a light input end including a light pulse spreading element;
a catheter sleeve including a capillary bundle assembly; and
a light output end, wherein said light output end comprises a compression element to create a compressed light pulse; a multiple light pulse combining element; a focusing element; a renewable target; and an exit window.

2. The catheter system of claim 1, wherein said stretching element receives the light pulse from a flashlamp-pumped solid-state laser.

3. The catheter system of claim 1, wherein said stretching element receives the light pulse from a diode-pumped solid-state laser.

4. The catheter system of claim 1, wherein said capillary bundle assembly includes a plurality of capillaries.

5. The catheter system of claim 1, wherein said light pulse spreading element receives the stretched light pulse and divides the stretched light pulse into multiple light pulses.

6. The catheter system of claim 5, wherein said multiple light pulses are each distributed to a capillary within said capillary bundle assembly.

7. The catheter system of claim 4, wherein each capillary within said capillary bundle assembly is coated with a high-damage-threshold material.

8. The catheter system of claim 4, wherein each capillary within the capillary bundle assembly guides a portion of a light pulse from the light input end to the light output end.

9. The catheter system of claim 7, wherein said high-damage-threshold material is a dielectric.

10. The catheter system of claim 7, wherein said high-damage-threshold material is a metal.

11. The catheter system of claim 1, wherein said compression element includes at least one of a chirped window, a transmission grating, a Bragg grating, and a dispersive medium to compress the light pulse.

12. The catheter system of claim 1, wherein said multiple light pulse combining element includes a lens array to combine the compressed light pulse from each capillary within the capillary bundle assembly into a single light pulse having a diameter larger than any compressed light pulse within a single capillary.

13. The catheter system of claim 1, wherein said multiple light pulse combining element combines the multiple light pulses from each capillary within said capillary bundle assembly prior to the light pulses being compressed by the compression element.

14. The catheter system of claim 1, wherein said focusing element increases intensity of the compressed light pulse by focusing the compressed light pulse.

15. The catheter system of claim 1, wherein said renewable target is capable of being ionized.

16. The catheter system of claim 1, wherein said renewable target is a patient's tissue.

17. The catheter system of claim 1, wherein said stretching element stretches said light pulse in time, wherein said stretched light pulse has a linear chirp and the same frequency bandwidth as the light pulse from the light input.

18. A method for treating tissue within a body having tissue including healthy and non-healthy tissue, the method comprising the steps of:
placing a catheter assembly within the body, said catheter assembly being part of a catheter system including a laser source for generating a light pulse; a stretching element for receiving the light pulse to form a stretched light pulse; wherein said catheter assembly includes a light input end including a light pulse spreading element; a catheter sleeve including a capillary bundle assembly and a light output end, wherein said light output end comprises a compression element to create a compressed light pulse; a multiple light pulse combining element; a focusing element; a renewable target; and an exit window;
generating the light pulse to be received by the stretching element and stretching the light pulse,
spreading the stretching light pulse with the light pulse spreading element such that a portion of the stretched light pulse is dispersed to each capillary within the capillary bundle and is propagated through the capillary bundle assembly;
combining the light pulse from each capillary of the capillary bundle assembly into a single light pulse with the multiple light pulse combining element to form a combined light pulse;
compressing the combined light pulse within the catheter assembly with the compression element to create a compressed light pulse;
focusing the compressed light pulse on said renewable target, thereby creating ionizing radiation;
applying the ionized radiation to non-healthy tissue.

19. The method of claim 18 wherein said non-healthy tissue is cancerous tissue.

20. The method of claim 18 wherein said non-healthy tissue is scar tissue formed as a result of restenosis.

21. A method for the detection of non-healthy tissue within a body having tissue including healthy and non-healthy tissue, the method comprising the steps of:
placing a catheter assembly within the body, said catheter assembly being part of a catheter system including a laser source for generating a light pulse; a stretching element for receiving the light pulse to form a stretched light pulse; wherein said catheter assembly includes a light input end including a light pulse spreading element; a catheter sleeve including a capillary bundle assembly and a light output end, wherein said light output end comprises a compression element to create a compressed light pulse; a multiple light pulse combining element; a focusing element; a renewable target; and an exit window;
generating the light pulse to be received by the stretching element and stretching the light pulse,
spreading the stretching light pulse with the light pulse spreading element such that a portion of the stretched light pulse is dispersed to each capillary within the capillary bundle and is propagated through the capillary bundle assembly;
combining the light pulse from each capillary of the capillary bundle assembly into a single light pulse with the multiple light pulse combining element to form a combined light pulse;
compressing the combined light pulse within the catheter assembly with the compression element to create a compressed light pulse;
focusing the combined light pulse on said renewable target, thereby creating ionizing radiation;
applying the ionized radiation within the body in conjunction with an external detector in order to determine whether the tissue in the body is tissue including cancerous tissue or healthy tissue.

22. An assembly for detecting structural damage in a structure, comprising
a laser source for generating a light pulse;
a stretching element for receiving the light pulse to form a stretched light pulse;
a detection assembly including
a light input end including a light pulse spreading element;
a detector sleeve including a capillary bundle assembly; and
a light output end, wherein said light output end comprises a compression element to create a compressed light pulse; a multiple light pulse combining element; a focusing element; a renewable target; and an exit window.

23. A method for detecting structural damage in a structure, comprising the steps of:
placing a detection assembly within the body, said detection assembly being part of a detection system including a laser source for generating a light pulse; a stretching element for receiving the light pulse to form a stretched light pulse; wherein said detection assembly includes a light input end including a light pulse spreading element; a detector sleeve including a capillary bundle assembly and a light output end, wherein said light output end comprises a compression element to create a compressed light pulse; a multiple light pulse combining element; a focusing element; a renewable target; and an exit window;
generating the light pulse to be received by the stretching element and stretching the light pulse,
spreading the stretching light pulse with the light pulse spreading element such that a portion of the stretched light pulse is dispersed to each capillary within the capillary bundle and is propagated through the capillary bundle assembly;
combining the light pulse from each capillary of the capillary bundle assembly into a single light pulse with the multiple light pulse combining element to form a combined light pulse;
compressing the stretched light pulse within the detector assembly with the compression element to create a compressed light pulse;
focusing the combined light pulse on the renewable target, thereby creating ionizing radiation;
applying the ionized radiation within the structure in conjunction with an external detector in order to detect whether there is structural damage in a structure.
